SEQUENCE LISTING (6 sequences)

SEQ ID NO: 1 - DNA, 864 bp, Artificial Sequence
  Description of Artificial Sequence Synthetic polynucleotide
atgcggggttctcatcatcatcatcatcatggtatggctagcatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgagggatccgagctcgagatctgcagcatgggctacgaccagctgccgccggggaaaaatccgcccgaagacatttacgtcgtaattgaaattcctcagggaagtgcggttaagtacgaacttgacaaagatacgggagttattttcgttgatcgtttcctgtttacggcgatgtactatccctttaattacggtttcgttccccagacgcttgccgacgacggagaccccgttgacgttcttgtcatatcaagagaacccgtagttcccggagcagttatgaggtgtagacccataggtatgctcgagatgagggacgaggcgggtatagacacgaaggtaatagcggttcctcacgaaaaactggacccctcctactcaaacattaagacagtggataacctccccgaaatagtcagagagaagataaaacacttctttgaacactacaaggaactcgaacccggaaagtgggtaaaagtggaaaactggaaaggacttcaggatgccatagaggagataaagaaagggattgaaaattacaagaaaaataaagaggggtaa

SEQ ID NO: 2 - protein, 287 aa, Artificial Sequence
  Description of Artificial Sequence Synthetic polypeptide
MRGSHHHHHHGMASMEAPAAAEISGHIVRSPMVGTFYRTPSPDAKAFIEVGQKVNVGDTLCIVEAMKMMNQIEADKSGTVKAILVESGQPVEFDEPLVVIEGSELEICSMGYDQLPPGKNPPEDIYVVIEIPQGSAVKYELDKDTGVIFVDRFLFTAMYYPFNYGFVPQTLADDGDPVDVLVISREPVVPGAVMRCRPIGMLEMRDEAGIDTKVIAVPHEKLDPSYSNIKTVDNLPEIVREKIKHFFEHYKELEPGKWVKVENWKGLQDAIEEIKKGIENYKKNKEG

SEQ ID NO: 3 - DNA, 864 bp, Artificial Sequence
  Description of Artificial Sequence Synthetic polynucleotide
atgcggggttctcatcatcatcatcatcatggtatggctagcatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatggcaatgatgaaccagatcgaagcggacaaatccggtaccgtgaaagcaattctggtcgaaagtggacaaccggtagaatttgacgagccgctggtcgtcatcgagggatccgagctcgagatctgcagcatgggctacgaccagctgccgccggggaaaaatccgcccgaagacatttacgtcgtaattgaaattcctcagggaagtgcggttaagtacgaacttgacaaagatacgggagttattttcgttgatcgtttcctgtttacggcgatgtactatccctttaattacggtttcgttccccagacgcttgccgacgacggagaccccgttgacgttcttgtcatatcaagagaacccgtagttcccggagcagttatgaggtgtagacccataggtatgctcgagatgagggacgaggcgggtatagacacgaaggtaatagcggttcctcacgaaaaactggacccctcctactcaaacattaagacagtggataacctccccgaaatagtcagagagaagataaaacacttctttgaacactacaaggaactcgaacccggaaagtgggtaaaagtggaaaactggaaaggacttcaggatgccatagaggagataaagaaagggattgaaaattacaagaaaaataaagaggggtaa

SEQ ID NO: 4 - protein, 287 aa, Artificial Sequence
  Description of Artificial Sequence Synthetic polypeptide
MRGSHHHHHHGMASMEAPAAAEISGHIVRSPMVGTFYRTPSPDAKAFIEVGQKVNVGDTLCIVEAMAMMNQIEADKSGTVKAILVESGQPVEFDEPLVVIEGSELEICSMGYDQLPPGKNPPEDIYVVIEIPQGSAVKYELDKDTGVIFVDRFLFTAMYYPFNYGFVPQTLADDGDPVDVLVISREPVVPGAVMRCRPIGMLEMRDEAGIDTKVIAVPHEKLDPSYSNIKTVDNLPEIVREKIKHFFEHYKELEPGKWVKVENWKGLQDAIEEIKKGIENYKKNKEG

SEQ ID NO: 5 - protein, 6 aa, Artificial Sequence
  Description of Artificial Sequence Synthetic 6xHis tag
HHHHHH

SEQ ID NO: 6 - protein, 7 aa, Artificial Sequence
  Description of Artificial Sequence Synthetic peptide
HHHHHHG

(19) United States
(12) Patent Application Publication
(10) Pub. No.: US 2012/0004115 A1
(43) Pub. Date: Jan. 5, 2012

(54) SYSTEM AND METHOD FOR PURIFICATION AND USE OF INORGANIC PYROPHOSPHATASE FROM AQUIFEX AEOLICUS

(75) Inventors: Kristian Hurd Link, Hamden, CT (US); Luan Pino, Branford, CT (US); Dawn Gratalo, East Haven, CT (US); Pitchai Sangan, Madison, CT (US); Suresh Gopalkrishna Shenoy, Branford, CT (US)

(73) Assignee: 454 Life Sciences Corporation

(21) Appl. No.: 13/097,580

(22) Filed: Apr. 29, 2011

Related U.S. Application Data
(60) Provisional application No. 61/329,795, filed on Apr. 30, 2010.

(51) Int. Cl. C40B 30/00; C12Q 1/68; C07H 21/04

(52) U.S. Cl. 506/7; 536/23.2; 435/6.1

(57) ABSTRACT

The present invention provides a nucleic acid that comprises a nucleic acid of SEQ ID NO: 1 or 3 encoding an Aae pyrophosphatase protein, an enzyme protein of SEQ ID NO: 2 or 4, and methods of sequencing using an isolated Aae pyrophosphatase protein.

### CROSS-REFERENCE TO RELATED APPLICATIONS

This application is related to and claims priority from U.S. Provisional Patent Application Ser. No. 61/329,795, titled “System and Method for Purification and Use of Inorganic Pyrophosphatase from Aquifex Aeolicus”, filed Apr. 30, 2010, which is hereby incorporated by reference herein in its entirety for all purposes.

### SEQUENCE LISTING

The instant application contains a Sequence Listing which has been submitted in ASCII format via EFS-Web and is hereby incorporated by reference in its entirety. The Sequence Listing, created on Apr. 28, 2011, is named 21467001.txt and is 8,654 bytes in size.

### FIELD OF THE INVENTION

The invention provides systems, methods, reagents, and kits for purification and use of inorganic pyrophosphatase enzyme. More specifically, the invention relates to the efficient isolation of inorganic pyrophosphatase enzyme and its uses in nucleic acid amplification and sequencing technologies.

### BACKGROUND OF THE INVENTION

Many amplification and sequencing strategies employ a polymerase enzyme for the addition of nucleotide species to newly synthesized nucleic acid molecules. It is generally appreciated that for each nucleotide species a polymerase incorporates, a Pyrophosphate molecule (also generally referred to as PPi) and a Hydrogen molecule is released into the reaction environment. This can be a very important consideration in amplification and sequencing strategies which employ very small reaction environments, because over many incorporation events by the polymerase, the PPi molecules accumulate in the incorporation events by the polymerase, the PPi molecules accumulate in the reaction environments, reaching concentrations where the PPi has an inhibitory effect upon the ability of the polymerase to incorporate nucleotide species.

Additionally, there are sequencing technologies that rely on the ability to detect the release of PPi. For example, measurements of the relative amounts of PPi released or a change in PPi concentration can be employed to indicate the incorporation of a nucleotide species that is complementary to a nucleotide species at a sequence position in a template molecule. The mode of detection or measurement can include changes in pH in the reaction environment, or via an enzyme cascade that produces a photon of light for each nucleotide molecule incorporated which is typically referred to as “Pyrosequencing”. In the present example, the degree of measured PPi is directly proportional to the number of nucleotide molecules incorporated and thus it is very important for the sequencing strategies described herein that the PPi detected during a nucleotide introduction step (i.e. a nucleotide flow discussed further below) is the result of release from incorporation of that particular nucleotide during that step and not a residual molecule from a previous step.

Therefore, strategies to reduce the concentration of PPi or remove it entirely from reaction environments are highly desirable in the described amplification and sequencing contexts. Typically, this can be accomplished via use of the PPi-ase enzyme which reacts with and specifically degrades PPi molecules. Previously identified versions of isolated PPi-ase enzyme reagent include a species derived from the Thermococcus litoralis bacterium and are available from New England Biolabs, Inc. (Also referred to as NEB, Ipswich Mass.). However, there is still a need for additional isolated PPi-ase enzyme reagent species that demonstrate characteristics desirable for use in amplification and sequencing technologies.

### SUMMARY OF THE INVENTION

Embodiments of the invention relate to the determination of the sequence of nucleic acids. More particularly, embodiments of the invention relate to methods and systems for correcting errors in data obtained during the sequencing of nucleic acids by sequencing by synthesis (SBS).

An embodiment of a nucleic acid is described that comprises a nucleic acid of SEQ ID NO: 1 or 3 encoding an Aae pyrophosphatase protein.

In addition, an embodiment of a method for sequencing using an isolated pyrophosphatase protein is described that comprises the steps of: performing a sequencing reaction in a reaction environment comprising an enzyme protein of SEQ ID NO: 2 or 4 derived from an Aquifex aeolicus species, wherein the enzyme protein comprises pyrophosphatase activity.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument under computer control and a reaction substrate;

FIG. 2 is a simplified graphical example of one embodiment of an Aquifex aeolicus pyrophosphatase fusion molecule (‘6-HIS’ disclosed as SEQ ID NO: 5);

FIGS. 3A and 3B are simplified graphical examples of a comparison of levels of activity of one embodiment of T. litoralis and one embodiment of Aquifex aeolicus PPi-ase enzymes;

FIG. 4 is a simplified graphical example of the thermostability demonstrated by one embodiment of Aquifex aeolicus PPi-ase;

FIGS. 5A and 5B are simplified graphical examples of a comparison of sequencing results obtained from E. coli on beads using one embodiment of T. litoralis and one embodiment of Aquifex aeolicus PPi-ase enzymes bound to beads;

FIGS. 6A and 6B are simplified graphical examples of a comparison of sequencing results obtained from C. jejuni on beads using one embodiment of T. litoralis and one embodiment of Aquifex aeolicus PPi-ase enzymes bound to beads; and

FIGS. 7A and 7B are simplified graphical examples of a comparison of sequencing results obtained from T. thermophilus on beads using one embodiment of T. litoralis and one embodiment of Aquifex aeolicus PPi-ase enzymes bound to beads.

### DETAILED DESCRIPTION OF THE INVENTION

As will be described in greater detail below, embodiments of the presently described invention include isolated nucleic acid sequences, protein sequences and/or products, expression systems, methods, and kits for purification and use of PPi-ase from the Aquifex aeolicus bacteria. In particular, embodiments of the invention relate to an isolated PPi-ase nucleic acid sequence coding for the PPi-ase enzyme and a fusion sequence derived therefrom comprising one or more elements that enable processing steps such as purification and/or biotinylation and are particularly useful for amplification of nucleic acid template molecules and for use in high throughput nucleic acid sequencing technology.

a. General

The term “flowgram” generally refers to a graphical representation of sequence data generated by SBS methods, particularly pyrophosphate based sequencing methods (also referred to as “pyrosequencing”) and may be referred to more specifically as a “pyrogram”.

The term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a serial or iterative cycle of addition of solution to an environment comprising a template nucleic acid molecule, where the solution may include a nucleotide species for addition to a nascent molecule or other reagent, such as buffers or enzymes that may be employed in a sequencing reaction or to reduce carryover or noise effects from previous flow cycles of nucleotide species.

The term “flow cycle” as used herein generally refers to a sequential series of flows where a nucleotide species is flowed once during the cycle (i.e. a flow cycle may include a sequential addition in the order of T, A, C, G nucleotide species, although other sequence combinations are also considered part of the definition). Typically, the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “test fragment” or “TF” as used herein generally refers to a nucleic acid element of known sequence composition that may be employed for quality control, calibration, or other related purposes.

The term “primer” as used herein generally refers to an oligonucleotide that acts as a point of initiation of DNA synthesis under conditions in which synthesis of a primer extension product complementary to a nucleic acid strand is induced in an appropriate buffer at a suitable temperature. A primer is preferably a single stranded oligodeoxyribonucleotide.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule.

The term “monomer repeat” or “homopolymers” as used herein generally refers to two or more sequence positions comprising the same nucleotide species (i.e. a repeated nucleotide species).

The term “homogeneous extension” as used herein, generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products, such as those produced from Polymerase Chain Reaction or Ligase Chain Reaction techniques.

The term “variant” or “allele” as used herein generally refers to one of a plurality of species each encoding a similar sequence composition, but with a degree of distinction from each other. The distinction may include any type of genetic variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations.

The term “allele frequency” or “allelic frequency” as used herein generally refers to the proportion of all variants in a population that is comprised of a particular variant.

The term “key sequence” or “key element” as used herein generally refers to a nucleic acid sequence element (typically of about 4 sequence positions, i.e., TGAC or other combination of nucleotide species) associated with a template nucleic acid molecule in a known location (i.e., typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (i.e., a “test fragment” or “TF” as referred to above) in a reaction well, where the accuracy of the sequence derived from TF sequence and/or Key sequence associated with the TF or in an adaptor associated with a target nucleic acid is compared to the known sequence composition of the TF and/or Key and used to measure of the accuracy of the sequencing and for quality control. In typical embodiments, a proportion of the total number of wells in a sequencing run will be keypass wells which may, in some embodiments, be regionally distributed.

The term “blunt end” as used herein is interpreted consistently with the understanding of one of ordinary skill in the related art, and generally refers to a linear double stranded nucleic acid molecule having an end that terminates with a pair of complementary nucleotide base species, where a pair of blunt ends are typically compatible for ligation to each other.

The term “sticky end” or “overhang” as used herein is interpreted consistently with the understanding of one of ordinary skill in the related art, and generally refers to a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”).

The term “SPRI” as used herein is interpreted consistently with the understanding of one of ordinary skill in the related art, and generally refers to the patented technology of “Solid Phase Reversible Immobilization” wherein target nucleic acids are selectively precipitated under specific buffer conditions in the presence of beads, where said beads are often carboxylated and paramagnetic. The precipitated target nucleic acids immobilize to said beads and remain bound until removed by an elution buffer according to the operator's needs (DeAngelis, Margaret M. et al: Solid-Phase Reversible Immobilization for the Isolation of PCR Products. Nucleic Acids Res (1995), Vol. 23:22; 4742-4743, which is hereby incorporated by reference herein in its entirety for all purposes).

The term “carboxylated” as used herein is interpreted consistently with the understanding of one of ordinary skill in the related art, and generally refers to the modification of a material, such as a microparticle, by the addition of at least one carboxl group. A carboxyl group is either COOH or COO—.

The term “paramagnetic” as used herein is interpreted consistently with the understanding of one of ordinary skill in the related art, and generally refers to the characteristic of a material wherein said material's magnetism occurs only in the presence of an external, applied magnetic field and does not retain any of the magnetization once the external, applied magnetic field is removed.

The term “bead” or “bead substrate” as used herein generally refers to any type of microparticle, wherein the term “microparticle” refers to any material of any convenient size, of irregular or regular shape and which is fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art.

The term “reaction environment” as used herein generally refers to a volume of space in which a reaction can take place typically where reactants are at least temporarily contained or confined allowing for detection of at least one reaction product. Examples of a reaction environment include but are not limited to cuvettes, tubes, bottles, as well as one or more depressions, wells, or chambers on a planar or non-planar substrate.

Some exemplary embodiments of systems and methods associated with sample preparation and processing, generation of sequence data, and analysis of sequence data are generally described below, some or all of which are amenable for use with embodiments of the presently described invention. In particular, the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample should be prepared and processed from its raw form into template molecules amenable for high throughput sequencing. The processing methods may vary from application to application, resulting in template molecules comprising various characteristics. For example, in some embodiments of high throughput sequencing, it is preferable to generate template molecules with a sequence or read length that is at least comparable to the length a particular sequencing method can accurately produce sequence data for. In the present example, the length may include a range of about 25-30 base pairs, about 50-100 base pairs, about 200-300 base pairs, about 350-500 base pairs, about 500-1000 base pairs, greater than 1000 base pairs, or other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will, however, be appreciated that other methods of fragmentation, such as digestion using restriction endonucleases, may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements (i.e. such as Key elements or other type of quality control element), unique identifiers (also referred to as a multiplex identifier or “MID”) that encode various associations such as with a sample of origin or patient, or other functional element.

For example, some embodiments of the described invention comprise associating one or more embodiments of an MID element having a known and identifiable sequence composition with a sample, and coupling the embodiments of MID element with template nucleic acid molecules from the associated samples. The MID coupled template nucleic acid molecules from a number of different samples are pooled into a single “Multiplexed” sample or composition that can then be efficiently processed to produce sequence data for each MID coupled template nucleic acid molecule. The sequence data for each template nucleic acid is de-convoluted to identify the sequence composition of coupled MID elements and association with sample of origin identified. In the present example, a multiplexed composition may include representatives from about 384 samples, about 96 samples, about 50 samples, about 20 samples, about 16 samples, about 12 samples, about 10 samples, or other number of samples. Each sample may be associated with a different experimental condition, treatment, species, or individual in a research context. Similarly, each sample may be associated with a different tissue, cell, individual, condition, drug or other treatment in a diagnostic context. Those of ordinary skill in the related art will appreciate that the numbers of samples listed above are for the purposes of example and thus should not be considered limiting.

In preferred embodiments, the sequence composition of each MID element is easily identifiable and resistant to introduced error from sequencing processes. Some embodiments of MID element comprise a unique sequence composition of nucleic acid species that has minimal sequence similarity to a naturally occurring sequence. Alternatively, embodiments of a MID element may include some degree of sequence similarity to naturally occurring sequence.

Also, in preferred embodiments the position of each MID element is known relative to some feature of the template nucleic acid molecule and/or adaptor elements coupled to the template molecule. Having a known position of each MID is useful for finding the MID element in sequence data and interpretation of the MID sequence composition for possible errors and subsequent association with the sample of origin.

For example, some features useful as anchors for positional relationship to MID elements may include, but are not limited to, the length of the template molecule (i.e. the MID element is known to be so many sequence positions from the 5′ or 3′ end), recognizable sequence markers such as a Key element and/or one or more primer elements positioned adjacent to a MID element. In the present example, the Key and primer elements generally comprise a known sequence composition that typically does not vary from sample to sample in the multiplex composition and may be employed as positional references for searching for the MID element. An analysis algorithm implemented by application 135 may be executed on computer 130 to analyze generated sequence data for each MID coupled template to identify the more easily recognizable Key and/or primer elements, and extrapolate from those positions to identify a sequence region presumed to include the sequence of the MID element. Application 135 may then process the sequence composition of the presumed region and possibly some distance away in the flanking regions to positively identify the MID element and its sequence composition.

Some or all of the described functional elements may be combined into adaptor elements that are coupled to nucleotide sequences in certain processing steps. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In some embodiments, two sets of priming sequence regions (hereafter referred to as priming sequence A, and priming sequence B) may be employed for strand selection, where only single strands having one copy of priming sequence A and one copy of priming sequence B is selected and included as the prepared sample. In alternative embodiments, design characteristics of the adaptor elements eliminate the need for strand selection. The same priming sequence regions may be employed in methods for amplification and immobilization where, for instance, priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. patent application Ser. No. 12/156,242, titled “System and Method for Identification of Individual Samples from a Multiplex Mixture”, filed May 29, 2008; and U.S. patent application Ser. No. 12/380,139, titled “System and Method for Improved Processing of Nucleic Acids for Production of Sequencable Libraries”, filed Feb. 23, 2009, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid, such as a water based fluid suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, or fluorinated oils.

Further, some emulsion embodiments may employ surfactants that act to stabilize the emulsion, which may be particularly useful for specific processing methods such as PCR. Some embodiments of surfactant may include one or more of a silicone or fluorinated surfactant. For example, one or more non-ionic surfactants may be employed that include, but are not limited to, sorbitan monooleate (also referred to as Span™ 80), polyoxyethylenesorbitsan monooleate (also referred to as Tween™ 80), or in some preferred embodiments, dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments, a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template and amplified copies of the template, amplified copies complementary to the template, or combination thereof. Further, the solid substrate may be enabled for attachment of other type of nucleic acids, reagents, labels, or other molecules of interest.

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. Pat. Nos. 7,638,276; 7,622,280; 7,842,457; and 7,927,797, each of which is hereby incorporated by reference herein in its entirety for all purposes.

Also embodiments sometimes referred to as Ultra-Deep Sequencing, generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants comprising sequence composition associated with a research or diagnostic utility where the primers may be employed to amplify and provide insight into the distribution of sequence variants in the sample. For example, a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (first amplicons) is subsequently further amplified individually in separate reaction vessels such as an emulsion based vessel described above. The resulting amplicons (referred to herein as second amplicons), each derived from one member of the first population of amplicons, are sequenced and the collection of sequences are used to determine an allelic frequency of one or more variants present. Importantly, the method does not require previous knowledge of the variants present and can typically identify variants present at <1% frequency in the population of nucleic acid molecules.

Some advantages of the described target specific amplification and sequencing methods include a higher level of sensitivity than previously achieved. Further, embodiments that employ high throughput sequencing instrumentation, such as for instance embodiments that employ what is referred to as a PicoTiterPlate® array (also sometimes referred to as a PTP™ plate or array) of wells provided by 454 Life Sciences Corporation, the described methods can be employed to generate sequence composition for over 100,000, over 300,000, over 500,000, or over 1,000,000 nucleic acid regions per run or experiment and may depend, at least in part, on user preferences such as lane configurations enabled by the use of gaskets, etc. Also, the described methods provide a sensitivity of detection of low abundance alleles which may represent 1% or less of the allelic variants. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; PCT Patent Application Serial No. US 2008/003424, titled “System and Method for Detection of HIV Drug Resistant Variants”, filed Mar. 14, 2008; and U.S. Pat. No. 7,888,034, titled “System and Method for Detection of HIV Tropism Variants”, filed Jun. 17, 2009, each of which is hereby incorporated by reference herein in its entirety for all purposes.

Further, embodiments of sequencing may include Sanger type techniques, techniques generally referred to as Sequencing by Hybridization (SBH), Sequencing by Ligation (SBL), or Sequencing by Incorporation (SBI) techniques. Further, the sequencing techniques may include what is referred to as polony sequencing techniques; nanopore, waveguide and other single molecule detection techniques; or reversible terminator techniques. As described above, a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) using an enzymatic reaction process to produce light or via detection of pH change (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include but are not limited to mass tags and fluorescent or chemiluminescent labels. In typical embodiments, unincorporated nucleotides are removed, for example by washing. Further, in some embodiments the unincorporated nucleotides may be subjected to enzymatic degradation such as, for instance, degradation using the apyrase or pyrophosphatase enzymes as described in U.S. patent application Ser. Nos. 12/215,455, titled “System and Method for Adaptive Reagent Control in Nucleic Acid Sequencing”, filed Jun. 27, 2008; and 12/322,284, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 500 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. Pat. No. 7,601,499, titled “Paired end sequencing”; and in U.S. patent application Ser. No. 12/322,119, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e., CCD camera) or confocal type architecture, a microfluidics chamber or flow cell, a reaction substrate, and/or a pump and flow valves. Taking the example of pyrophosphate based sequencing, embodiments of an apparatus may employ a chemiluminescent detection strategy that produces an inherently low level of background noise.

In some embodiments, the reaction substrate for sequencing may include a planar substrate such as a slide type substrate, an Ion-Sensitive Field Effect Transistor (also referred to as “ISFET”), or waveguide type reaction substrate that in some embodiments may comprise well type structures. Further the reaction substrate may include what is referred to as a PTP™ array available from 454 Life Sciences Corporation, as described above, formed from a fiber optic faceplate that is acid-etched to yield hundreds of thousands or more of very small wells each enabled to hold a population of substantially identical template molecules (i.e., some preferred embodiments comprise about 3.3 million wells on a 70×75 mm PTP™ array at a 35 μm well to well pitch). In some embodiments, each population of substantially identical template molecule may be disposed upon a solid substrate, such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. Pat. No. 7,682,816, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. Nos. 7,323,305 and 7,575,865, both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. Pat. No. 7,927,797, titled “Nucleic acid amplification with continuous flow emulsion”, filed Jan. 28, 2005, which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Celeron®, Core™, or Pentium® processor made by Intel Corporation, a SPARC® processor made by Sun Microsystems, an Athlon™, Sempron™, Phenom™, or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP, Windows Vista®, or Windows®_{—}7) from the Microsoft Corporation; the Mac OS X operating system from Apple Computer Corp. (such as Mac OS X v10.6 “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example, some commonly employed web browsers include Microsoft® Internet Explorer 8 available from Microsoft Corporation, Mozilla Firefox® 3.6 from the Mozilla Corporation, Safari 4 from Apple Computer Corp., Google Chrome from the Google™ Corporation, or other type of web browser currently known in the art or to be developed in the future. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

b. Embodiments of the Presently Described Invention

As described above embodiments of the described invention are directed to improved systems, methods, and kits associated with Aquifex aeolicus (also sometimes referred to as “Aae”) PPi-ase and its uses. Those of ordinary skill in the related art will appreciate that Aquifex aeolicus is a thermophilic bacteria typically found near underwater volcanoes or hot springs where water temperatures may reach 85-95° C. An isolated PPi-ase enzyme produced by Aquifex aeolicus has been described by Hoe et al (Hyang-Sook Hoe, Hyun-Kyu Kim, Suk-Tae Kwon, Expression in Escherichia coli of the Thermostable Inorganic Pyrophosphatase from the Aquifex aeolicus and Purification and Characterization of the Recombinant Enzyme, Protein Expression and Purification, Vol 23, Issue 2, November 2001, Pages 242-248) and demonstrated a very high level of heat stability and efficiency at elevated temperatures which are traits that are generally appreciated to be advantageous for PCR and particular sequencing applications. The presently described invention includes nucleotide and protein sequences that encode a PPi-ase enzyme isolated from Aquifex aeolicus that resists denaturation at temperatures commonly employed in PCR and sequencing technologies as well as having significant enzyme activity at said temperatures. Also, embodiments of the invention are described that include one or more additional functional elements that enable further modifications to and/or improve processing efficiency of the protein.

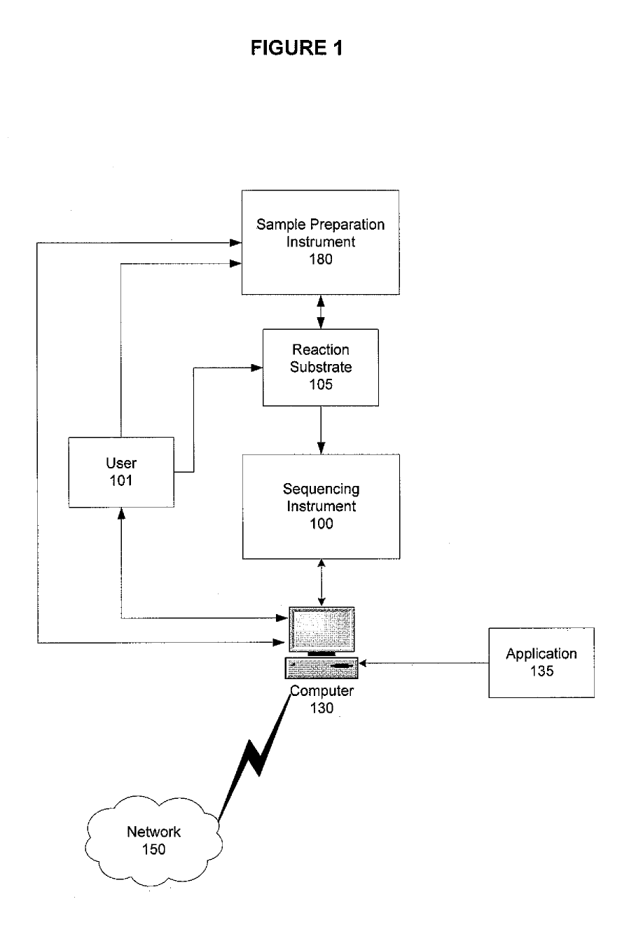

In a typical sequencing embodiment one or more instrument elements may be employed that automate one or more process steps. For example, embodiments of a sequencing method may be executed using instrumentation to automate and carry out some or all process steps. FIG. 1 provides an illustrative example of sequencing instrument 100 that for sequencing processes requiring capture of optical signals typically comprise an optic subsystem and a fluidic subsystem for execution of sequencing reactions and data capture that occur on reaction substrate 105. It will, however, be appreciated that for sequencing processes requiring other modes of data capture (i.e. pH, temperature, electrochemical, etc.) a subsystem for the mode of data capture may be employed which are known to those of ordinary skill in the related art. For instance, a sample of template molecules may be loaded onto reaction substrate 105 by user 101 or some automated embodiment, then sequenced in a massively parallel manner using sequencing instrument 100 to produce sequence data representing the sequence composition of each template molecule. Importantly, user 101 may include any such user that includes but is not limited to an independent researcher, technician, clinician, university, or corporate entity.

Embodiments of sequencing instrument 100 employed to execute sequencing processes may include various fluidic components in the fluidic subsystem, various optical components in the optic subsystem, as well as additional components not illustrated in FIG. 1 that may include microprocessor and/or microcontroller components for local control of some functions. In some embodiments samples may be optionally prepared for sequencing in an automated or partially automated fashion using sample preparation instrument 180 configured to perform some or all of the necessary preparation for sequencing using instrument 100. Further, as illustrated in FIG. 1 sequencing instrument 100 may be operatively linked to one or more external computer components such as computer 130 that may for instance execute system software or firmware such as application 135 that may provide instructional control of one or more of the instruments such as sequencing instrument 100 or sample preparation instrument 180, and/or data analysis functions. Computer 130 may be additionally operatively connected to other computers or servers via network 150 that may enable remote operation of instrument systems and the export of large amounts of data to systems capable of storage and processing. In the present example, sequencing instrument 100 and/or computer 130 may include some or all of the components and characteristics of the embodiments generally described above.

As described above one aspect of the described invention includes a nucleic acid sequence encoding an Aae PPi-ase and corresponding amino acid sequence. As described above, in some embodiments it is also advantageous to add other functional elements to improve processing and isolation of the enzyme protein. One particularly useful strategy is to include elements enabling in-vivo biotinylation of the enzyme protein. Those of ordinary skill in the art will appreciate that biotin is a very useful molecular biology tool for preferential isolation of elements of interest such as nucleic acids, protein, substrates, etc. and further that typically in-vitro based methods are employed to associate one or more biotin elements with a protein or nucleic acid which generally require more processing steps and thus are less efficient that the in-vivo methods described herein. In some embodiments, the use of biotin is useful to sequester target molecules, such as for instance Aae PPi-ase enzyme proteins, to a substrate which can be used in sequencing processes executed on a substrate comprising a plurality of individual reaction environments such as the PTP substrate described above. For example, Aae PPi-ase may preferably be biotinylated in order to interact with and bind to a bead substrate such as a Magnosphere MS300 Streptavidin coated bead available from JSR Corporation.

It will be appreciated however, that for some applications biotinylation may not be desirable. For example, it may not be desirable to employ biotinylated PPi-ase in emPCR processes described above, or for use a reagent introduced in a flow during a sequencing flow cycle. However, in the present example the biotinylated PPi-ase enzyme could still be used in said processes.

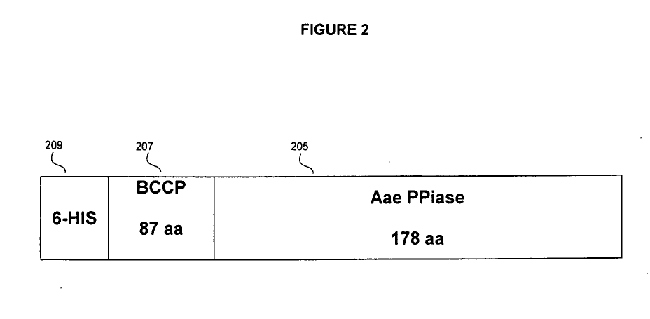

One means of enabling in-vivo biotinylation can be accomplished through the incorporation of a biotin carboxyl carrier protein (also referred to as BCCP) domain into a fusion sequence. Other elements may also be included such as a 6-histidine moiety (also referred to as a His tag) (SEQ ID NO: 5) that further enables “one-step” purification using an affinity column (i.e. such as a Ni²⁺ affinity column). FIG. 2 provides an illustrative example of one possible configuration of Aae PPi-ase and other functional elements such as PPi-ase 205, BCCP 207, and H is 209. For example, as those of ordinary skill in the art appreciate an affinity tag may be associated with a molecule via use of a BCCP domain which provides a site for in-vivo expression and biotinylation of the protein by E. coli ligase. In the present example, a plasmid comprising the nucleic acid sequence encoding Aae PPi-ase and associated functional elements may be transformed into E. coli cells and grown in media to produce many copies. The cells can then be harvested and lysed and the expressed proteins collected using affinity columns which recognize the His tag.

In some embodiments the BCCP domain may also include a “point mutation” at a single sequence position which inhibits biotinylation of the protein product. For example, the BCCP domain may comprise a point mutation that changes a lysine amino acid to an alanine, which prevents biotinylation producing a protein which may be more amenable for use in embodiments where solution phase PPi-ase is more desirable.

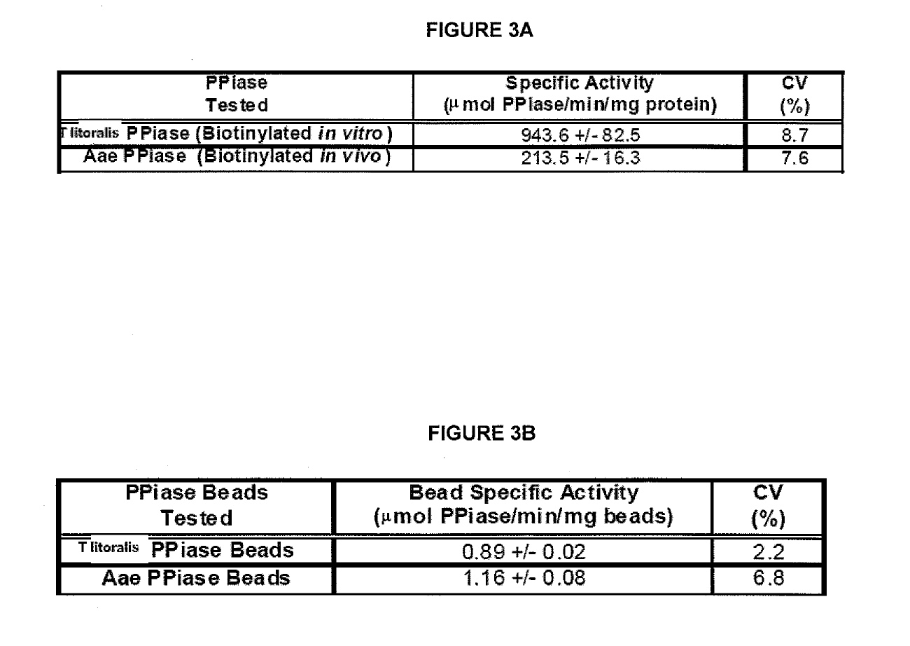

FIGS. 3A and 3B provide illustrative examples of a comparison of enzyme activity of T. litoralis PPi-ase which was biotinylated in-vitro to an embodiment of Aae PPi-ase which was biotinylated in-vivo, where the specific activity of the Aae PPi-ase protein immobilized on bead substrates is equal to or greater than the specific activity of the bead immobilized T. litoralis PPi-ase protein.

Embodiments of the invention may include one or more of the following sequences:

SEQ ID NO: 1: Nucleotide sequence that
encodes the Aae-BCCP fusion protein.
ATGCGGGGTTCTCATCATCATCATCATCATGGTATGGCTAGCAT
GGAAGCGCCAGCAGCAGCGGAAATCAGTGGTCACATCGTACGTT
CCCCGATGGTTGGTACTTTCTACCGCACCCCAAGCCCGGACGCA
AAAGCGTTCATCGAAGTGGGTCAGAAAGTCAACGTGGGCGATAC
CCTGTGCATCGTTGAAGCCATGAAAATGATGAACCAGATCGAAG
CGGACAAATCCGGTACCGTGAAAGCAATTCTGGTCGAAAGTGGA
CAACCGGTAGAATTTGACGAGCCGCTGGTCGTCATCGAGGGATC
CGAGCTCGAGATCTGCAGCATGGGCTACGACCAGCTGCCGCCGG
GGAAAAATCCGCCCGAAGACATTTACGTCGTAATTGAAATTCCT
CAGGGAAGTGCGGTTAAGTACGAACTTGACAAAGATACGGGAGT
TATTTTCGTTGATCGTTTCCTGTTTACGGCGATGTACTATCCCT
TTAATTACGGTTTCGTTCCCCAGACGCTTGCCGACGACGGAGAC
CCCGTTGACGTTCTTGTCATATCAAGAGAACCCGTAGTTCCCGG
AGCAGTTATGAGGTGTAGACCCATAGGTATGCTCGAGATGAGGG
ACGAGGCGGGTATAGACACGAAGGTAATAGCGGTTCCTCACGAA
AAACTGGACCCCTCCTACTCAAACATTAAGACAGTGGATAACCT
CCCCGAAATAGTCAGAGAGAAGATAAAACACTTCTTTGAACACT
ACAAGGAACTCGAACCCGGAAAGTGGGTAAAAGTGGAAAACTGG
AAAGGACTTCAGGATGCCATAGAGGAGATAAAGAAAGGGATTGA
AAATTACAAGAAAAATAAAGAGGGGTAA
SEQ ID NO. 2: Amino acid sequence of the
Aae-BCCP fusion protein
MRGSHHHHHHGMASMEAPAAAEISGHIVRSPMVGTFYRTPSPDA
KAFIEVGQKVNVGDTLCIVEAMKMMNQIEADKSGTVKAILVESG
QPVEFDEPLVVIEGSELEICSMGYDQLPPGKNPPEDIYVVIEIP
QGSAVKYELDKDTGVIFVDRFLFTAMYYPFNYGFVPQTLADDGD
PVDVLVISREPVVPGAVMRCRPIGMLEMRDEAGIDTKVIAVPHE
KLDPSYSNIKTVDNLPEIVREKIKSIFFEHYKELEPGKWVKVEN
WKGLQDAIEEIKKGIENYKKNKEG
SEQ ID NO: 3: Nucleotide sequence that
encodes the Aae-BCCP mutant fusion protein.
ATGCGGGGTTCTCATCATCATCATCATCATGGTATGGCTAGCAT
GGAAGCGCCAGCAGCAGCGGAAATCAGTGGTCACATCGTACGTT
CCCCGATGGTTGGTACTTTCTACCGCACCCCAAGCCCGGACGCA
AAAGCGTTCATCGAAGTGGGTCAGAAAGTCAACGTGGGCGATAC
CCTGTGCATCGTTGAAGCCATGgcAATGATGAACCAGATCGAAG
CGGACAAATCCGGTACCGTGAAAGCAATTCTGGTCGAAAGTGGA
CAACCGGTAGAATTTGACGAGCCGCTGGTCGTCATCGAGGGATC
CGAGCTCGAGATCTGCAGCATGGGCTACGACCAGCTGCCGCCGG
GGAAAAATCCGCCCGAAGACATTTACGTCGTAATTGAAATTCCT
CAGGGAAGTGCGGTTAAGTACGAACTTGACAAAGATACGGGAGT
TATTTTCGTTGATCGTTTCCTGTTTACGGCGATGTACTATCCCT
TTAATTACGGTTTCGTTCCCCAGACGCTTGCCGACGACGGAGAC
CCCGTTGACGTTCTTGTCATATCAAGAGAACCCGTAGTTCCCGG
AGCAGTTATGAGGTGTAGACCCATAGGTATGCTCGAGATGAGGG
ACGAGGCGGGTATAGACACGAAGGTAATAGCGGTTCCTCACGAA
AAACTGGACCCCTCCTACTCAAACATTAAGACAGTGGATAACCT
CCCCGAAATAGTCAGAGAGAAGATAAAACACTTCTTTGAACACT
ACAAGGAACTCGAACCCGGAAAGTGGGTAAAAGTGGAAAACTGG
AAAGGACTTCAGGATGCCATAGAGGAGATAAAGAAAGGGATTGA
AAATTACAAGAAAAATAAAGAGGGGTAA
SEQ ID NO: 4: Amino acid sequence of the
Aae-BCCP mutant fusion protein
MRGSHHHHHHGMASMEAPAAAEISGHIVRSPMVGTFYRTPSPDA
KAFIEVGQKVNVGDTLCIVEAMAMMNQIEADKSGTVKAILVESG
QPVEFDEPLVVIEGSELEICSMGYDQLPPGKNPPEDIYVVIEIP
QGSAVKYELDKDTGVIFVDRFLFTAMYYPFNYGFVPQTLADDGD
PVDVLVISREPVVPGAVMRCRPIGMLEMRDEAGIDTKVIAVPHE
KLDPSYSNIKTVDNLPEIVREKIKHFFEHYKELEPGKWVKVENW
KGLQDAIEEIKKGIENYKKNKEG

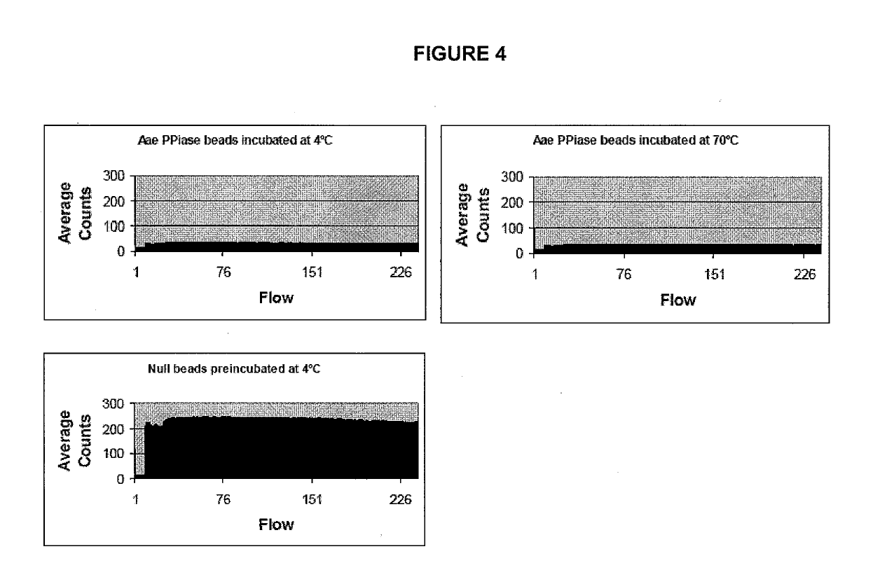

As described above, a highly desirable characteristic of the Aae PPi-ase protein described herein is its thermostability an example of which is illustrated in FIG. 4 where PPi was introduced into an array substrate comprising a large number of well reaction environments comprising Aae PPi-ase immobilized upon bead substrates along with other necessary reactants required to produce light (e.g. sulfurylase, APS, luciferase, and D-luciferin). As demonstrated in the example of FIG. 4, beads that did not have Aae PPi-ase immobilized (i.e. “null”) showed a significantly higher detected light signal for more than 226 flows than beads having immobilized Aae PPi-ase that were incubated at 4° C. and 70° C. In other words, the immobilized Aae PPi-ase incubated at high temperature retained its enzymatic activity and efficiently degraded the introduced PPi-ase at least 226 times so that relatively little PPi was available after each flow to produce light.

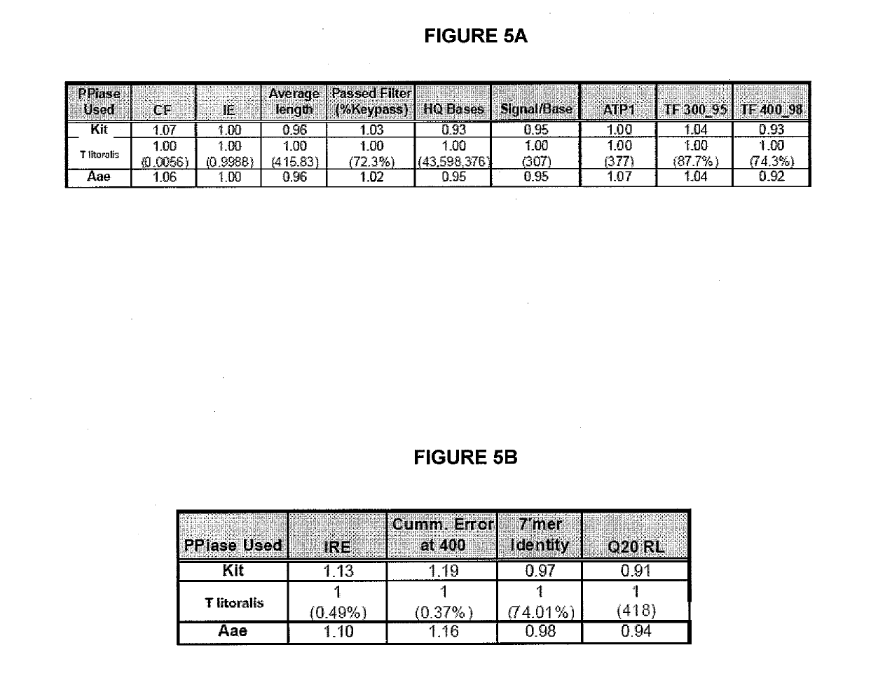

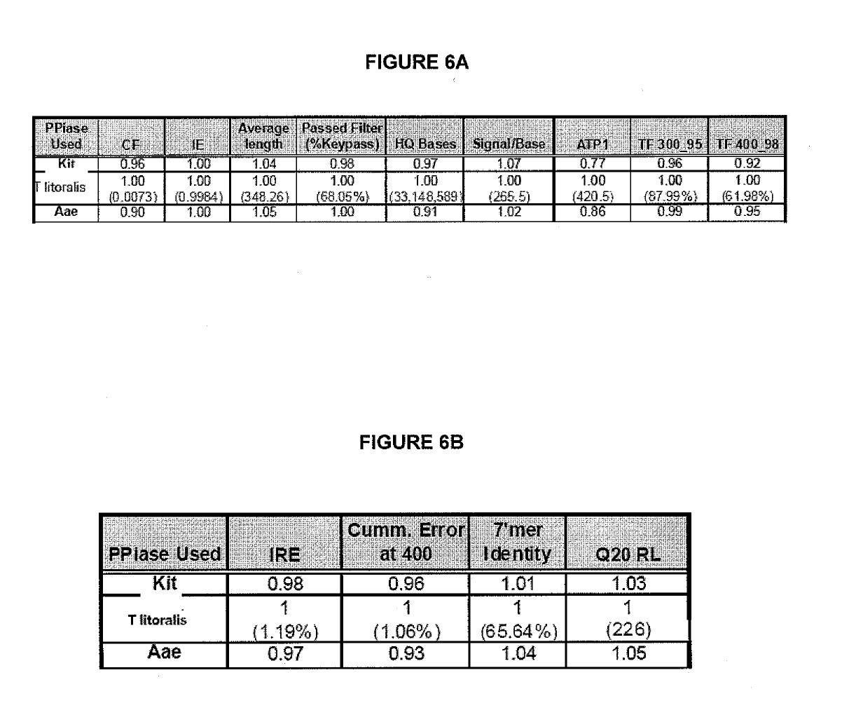

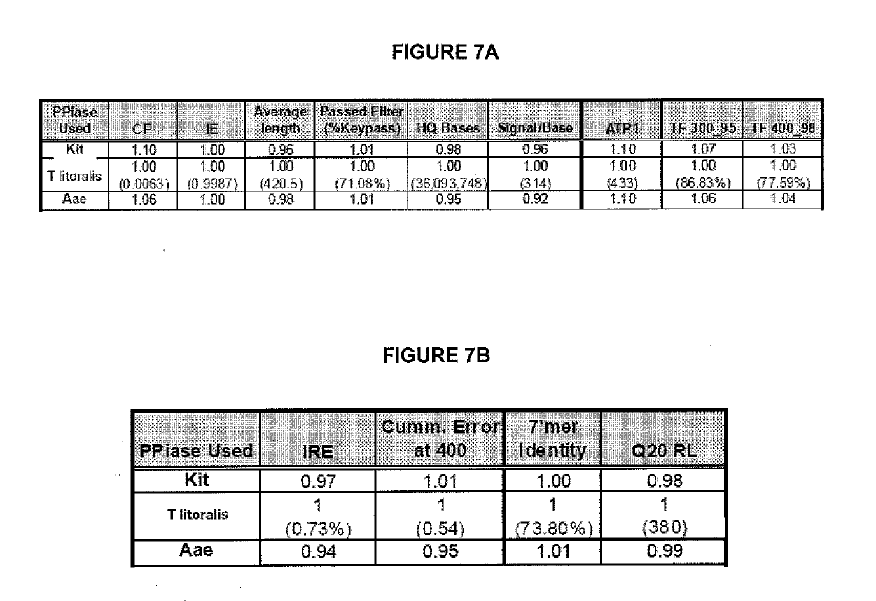

Further, FIGS. 5-7 provides illustrative examples of sequencing results obtained using bead immobilized T. litoralis and Aae PPi-ase in a PIP array of well reaction environments for E. coli (FIG. 5), C. jejuni (FIG. 6), and T thermophilus (FIG. 7) which each have different sequence composition characteristics. It will be appreciated by those of ordinary skill that the numbers are normalized to T. litoralis (i.e. the T. litoralis numbers are 1 in each category) and it is important to note that in each case the Aae PPi-ase provides a level of performance that is substantially the same as T litoralis PPi-ase.

It will also be appreciated by those of ordinary skill in the art that embodiments which employ bead bound Aae PPi-ase in array substrates comprising high numbers of well type reaction environments in close proximity to one another typically have reduced well to well diffusion of PPi reaction products from those embodiments which do not use PPi-ase in the wells as described in U.S. patent application Ser. No. 12/322,284 which is incorporated by reference above.

### EXAMPLES

### Example 1

### Expression of the Thermostable Inorganic Pyrophosphatase from Aquifex Aeolicus in E. coli

Day 1: Preparing Freshly Transformed Cells

The pRSET-6HIS-BCCP-Aae plasmid (‘6HIS’ disclosed as SEQ ID NO: 5) was diluted 100-fold in dH₂O (e.g., 1 μL stock plasmid plus 99 μL water, then vortexed to mix the solution. Three tubes of One Shot BL21(DE3)pLysS chemically-competent cells were removed from −80° C. and placed on ice, where they were allowed to thaw on ice for 10 min. The diluted plasmid (1 μL) was added to two of the tubes. The third tube was a control tube. The tubes were gently tapped on a flat surface and incubated on ice for 30 minutes. A heat block containing the correct holder for 1.7 mL microcentrifuge tubes was set to 42° C. and all three tubes (two with plasmid and one control) were heat shocked by incubating the tubes in the heat block for 30 seconds at 42° C. The cells were then incubated on ice for 2 minutes.

Two hundred and fifty microliters of room temperature SOC media were added to each tube and the tubes placed into a tube rack with a strip of tape across their lids to secure them for horizontal shaking. The tubes were incubated for 1 hour in an orbital shaker at 37° C., 250 rpm. The cells (100 μL) were plated onto LB+Amp+Cam plates from each of the tubes using a cell spreader. One plate was used for each tube of cells. The plates were then incubated upside down at 37° C., overnight.

Day 2: Overnight Culture

To a 1 liter Erlenmeyer flask, 200 mL of room temperature LB, 200 μL of 100 mg/mL Amp and 200 μL of 34 mg/mL chloramphenicol were added. Using a sterile tooth pick, individual colonies were transferred into each of the flasks containing media. In some instances, an inoculating loop was used to transfer cells from a glycerol stock into the Erlenmeyer flask (1 L) containing media. The Erlenmeyer flask was incubated overnight in an orbital shaker at 37° C., 250 rpm.

Day 3: Starter Culture

To an appropriately sized Erlenmeyer flask, 900 mL of room temperature LB, 1 mL of 100 mg/mL Amp, 1 mL of 34 mg/mL chloramphenicol were added. Nine hundred milliliters of room temperature LB, 1 mL of 100 mg/mL Amp, 1 mL of 34 mg/mL chloramphenicol were added to a second Erlenmeyer flask. Each Erlenmeyer flask was inoculated with 100 mL of the overnight culture and labeled. The Erlenmeyer flasks were then incubated in an orbital shaker at 37° C., 250 rpm until the OD₆₀₀ was approximately 0.7 (approximately 3 hours). The OD₆₀₀ was not permitted to increase greater than 1.0 before induction.

Induction

One milliliter from the Erlenmeyer flasks was withdrawn and transferred to individual 1.5 mL microcentrifuge tubes that were previously marked with “t=0” and the parent Erlenmeyer flask number. Induction was commenced by adding 1 mL of 1 M IPTG and 12 mg of biotin powder into each Erlenmeyer flask. The final concentration of biotin (FW 244.3 g/mol) in each 1 L culture was 50 μM. The Erlenmeyer flasks were incubated in an orbital shaker at 37° C., 250 rpm for an additional 3 hours. During this time, the buffers for the PPiase purification were prepared. After induction was complete, the OD₆₀₀ of each culture was measured. One milliliter of the solution from each Erlenmeyer flask were withdrawn and transferred to individual 1.5 mL microcentrifuge tubes that were previously marked with “t=3” and the parent Erlenmeyer flask number.

Harvesting Cells

The cells of the t=0 and t=3 time points were pelleted in the microcentrifuge tubes at 10,000 RCF for 10 min in a bench top centrifuge. The supernatant was removed without disturbing the pellet. The tubes were stored at −80° C. for later analysis by standard SDS-PAGE (Invitrogen) and Western blot analysis (Invitrogen) using an anti-6HisGly (SEQ ID NO: 6) primary antibody (Invitrogen) and an appropriate secondary antibody. The mass of 2 to 4 empty centrifuge bottles was obtained. The 2 L culture volume was pelleted using 1 or 2 centrifuge bottle per Erlenmeyer flasks in a pre-chilled SLA-3000 rotor at 4° C., 5,000 RCF for 10 min using a Sorvall RC-5B centrifuge. Collection was performed repetitively by decanting the cleared supernatant and adding more culture to the centrifuge bottles until all the cells were pelleted. The mass of the centrifuge bottles plus cell pellet was obtained. The difference in mass between this mass and the mass obtained in step 25 above constitutes the mass of the cells. Approximately 4.5 g of cell mass was obtained per liter of culture. The centrifuge bottles were then marked with colored tape containing the date, initials and contents. The tubes were stored at −80° C. until needed for the enzyme purification

### Example 2

### Purification of the Biotinylated Thermostable Inorganic Pyrophosphatase from Aquifex aeolicus

Charging and Equilibrating the Column

The appropriate tubing was connected to a peristaltic pump. The outlet end of the peristaltic pump tubing was connected to the inlet end of a 5 mL HiTrap chelating HP column (GE Health Care). The inlet end of the peristaltic pump tubing was placed into a large beaker full of ˜1 L dH₂O (at ambient temperature). The tubing was connected to the outlet end of the column and placed in a waste reservoir. The flow was started at 1 mL/min for 10 CV. The chelating resin was charged with Ni²⁺ by pumping 20 ml of 0.1 M NiSO₄ at 1 mL/min into the column. The unchelated Ni²⁺ was washed out with dH₂O, 1 mL/min, 5 CV. The column was moved to the 4° C. refrigerator and allowed to equilibrate for at least 1 hour before commencing any additional flows. The affinity column with 5 CV of buffer A was equilibrated at a flow rate of 1

Lysis and Clarification

The net weight of the frozen cell pellets from the 6His-BCCP-Aae PPiase (‘6His’ disclosed as SEQ ID NO: 5) expression procedure was determined. The pellet(s) were thawed on ice for 30 minutes. During this time, the lysis solution was prepared, in an amount of 5 ml for every gram of pelleted cells, to a maximum of 40 mL.

Lysis Solution:

| | | Quantity | | | Reference |
|---|---|---|---|---|---|
| Reagent | Stock Conc | Required | | Source | Number |
| BugBuster | 10X | 2 | mL | Novagen | 70921-4 |
| solution | | | | | |
| PBS | 10X | 2 | mL | MP | 1960454 |
| | | | | Biomedicals | |
| MgCl₂ | 1M | 20 | μL | Ambion | 9530G |
| Benzonase | 10,000 | 50 | μL | Novagen | 70664-3 |
| | units/mL | | | | |
| 100x Protease | 10X | 200 | μL | Fisher | 78415 |
| Inhibitor | | | | | |
| Cocktail, | | | | | |
| EDTA free | | | | | |

The above reagents were combined and adjusted to V_{f}=20 ml with dH₂O. The lysis solution contained 1× BugBuster, 1×PBS, 25 U/ml Benzonase and 1 mM MgCl₂. The lysis solution was added to the cell pellet in 5 ml aliquots and the pellet resuspended by gently passing clumps up-and-down a 10 ml graduated pipet using a Pipet-Aid. Once the clumps were dispersed and all of the lysis solution was added, the tube was capped and placed on a Nutator for 15 min at room temperature. The SLA-3000 rotor was placed in the Sorvall centrifuge and chilled to 4° C.

The lysate was diluted 4-fold with 3 volumes of Buffer A (Buffer A contains 1×PBS, 0.5 M NaCl, and 10 mM imidazole. The components were mixed, adjusted to Vf=1 L with dH2O, filtered with a 0.2 μm Stericup, and stored at 4° C. The centrifuge bottles were loaded in balanced pairs (tolerance<0.2 g) and centrifuged in the SLA-3000 rotor at 9,000 rpm, 4° C. for 20 minutes. At the end of the centrifuge spin, the supernatant (“clarified lysate”) of all tubes was decanted into a single flask or beaker. The supernatant was retained as it contained soluble protein. The combined supernatants were swirled and placed on ice.

Affinity Purification

The clarified lysate was loaded onto the affinity column at 1 mL/min flow rate via the peristaltic pump. The flow through was collected as a single fraction. The column was washed with 7 CV of Buffer A at 1 mL/min flow rate via the peristaltic pump. The flow through was collected as a single fraction. The inlet of the column was disconnected from the peristaltic pump and connected to the outlet of a gradient mixer with appropriate reservoir size. A stir bar was placed into the chamber connected to the outlet of the gradient mixer. The gradient mixer was placed onto a magnetic stir plate.

The chamber connected to the outlet was filled with 5 CV of Buffer A, while the other chamber was filled with 5 CV of Buffer B (Buffer B contains 1×PBS, 0.5 M NaCl, and 500 mM imidazole. All components were mixed and adjusted to V_{f}=1 L with dH₂O, then filtered with a 0.2 μM Stericup and stored at 4° C. The stir plate was used to allow for efficient mixing of the buffer within the changer.

The protein was then eluted from the affinity column by opening the outlet of the gradient mixer, allowing the buffer to flow onto the column. One milliliter fractions were collected. The inlet of the column was disconnected from the peristaltic pump and connected to fresh tubing. The inlet end of the peristaltic pump tubing was placed into a large beaker full of Buffer B and flow was commenced at 1 mL/min for 4 CV. One milliliter fractions were collected.

After confirmation that the protein eluted from the column, the column was washed and stored at 4° C. Washing the affinity column at 1 mL/min via the peristaltic pump was achieved as follows:

a. 5 CV CIP

b. 10 CV dH₂O

c. 2 CV 20% EtOH

Pooling fractions, dialysis and storage.

The fractions were analyzed by standard SDS-PAGE (Invitrogen). The 6-His BCCP-Aae PPiase (‘6His’ disclosed as SEQ ID NO: 5) protein has a molecular weight of approximately 32 kDa. The pooled fractions were loaded into the appropriate number of 10K MWCO Slide-A-Lyzer dialysis units which were pre-wetted with PPiase storage buffer.

PPiase Storage Buffer:

| | | Quantity | | | Reference |
|---|---|---|---|---|---|
| Reagent | Stock Conc | Required | | Source | Number |
| Tricine, pH | 1M | 40 | mL | 454 Life | 0000701 |
| 7.8 | | | | Sciences | |
| KCl | 1M | 200 | mL | TEKnova | P0325 |
| DTT | 1M | 2 | mL | 454 Life | 0000725 |
| | | | | Sciences | |
| Glycerol | NA | 1 | L | Fisher | BP229-1 |

The dialysis and storage buffer is 50 mM Tricine (pH 7.8), 100 mM KCl, 1 mM DTT and 50% glycerol. All components were mixed, adjusted to V_{f}=2 L with dH₂O, filtered with a 0.2 μm Stericup and stored at 4° C. The 1 M Tricine buffer (pH 7.8) was confirmed to have low PPi background. The Slide-A-Lyzer dialysis units were inserted into a carousel and placed in 2 L of PPiase storage buffer at 4° C. The units were incubated overnight at 4° C. while stirring. The following day, the dialysis buffer were replaced with 2 L of fresh PPiase storage buffer at 4° C., then incubated overnight at 4° C. while stirring. The next day, the retentate(s) from the dialysis Slide-A-Lyzer dialysis unit(s) was recovered.

The protein concentration of the solution was measured using the Bio-Rad protein assay kit, using BSA as the standard. The purification yielded a total of around 91 mg of protein. The purity of the sample was determined by standard SDS-PAGE (Invitrogen) and Western blot analysis (Invitrogen) using an anti-6HisGly (SEQ ID NO: 6) primary antibody (Invitrogen) and an appropriate secondary antibody. The protein was stored at −80° C.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. A nucleic acid, comprising:
a nucleic acid of SEQ ID NO: 1 or 3 encoding an Aae pyrophosphatase enzyme.

2. The nucleic acid of claim 1, wherein:
the nucleic acid encodes a His tag.

3. The nucleic acid of claim 1, wherein:
the nucleic acid encodes a BCCP biotinylation site.

4. A method for sequencing using an isolated pyrophosphatase protein comprising the steps of:
performing a sequencing reaction in a reaction environment comprising an enzyme protein of SEQ ID NO: 2 or 4 derived from an Aquifex aeolicus species, wherein the enzyme protein comprises pyrophosphatase activity.

5. The method of claim 4, wherein:
the enzyme protein is bound to a bead.

6. The method of claim 5, wherein:
the enzyme protein is bound to the bead by a biotin linkage.

7. The method of claim 6, wherein:
the biotin is operatively coupled to the protein using an in-vivo process.

8. The method of claim 4, wherein:
the enzyme protein is thermostable.

9. The method of claim 4, wherein:
a plurality of the sequencing reactions are performed in a plurality of the reaction environments simultaneously.
